(19) United States
(12) Patent Application Publication
(10) Pub. No.: US 2006/0275247 A1
(43) Pub. Date: Dec. 7, 2006

(54) Cosmetic Compositions With Moringa Seed Extract

(75) Inventors: Michael David Helman, Edison, NJ (US); Christopher James Cashin, Dunellen, NJ (US); Gale McElroy Reinhart, Middletown, NJ (US)

Correspondence Address: JULIE BLACKBURN;REVLON CONSUMER PRODUCTS CORPORATION, 237 PARK AVENUE, NEW YORK, NY, 10017, US

(73) Assignee: Revlon Consumer Products Corporation

(21) Appl. No.: 11/380,428

(22) Filed: Apr. 27, 2006

Related U.S. Application Data
(60) Provisional application No. 60/686,208, filed on Jun. 1, 2005.

(51) Int. Cl. A61K 8/97; A61K 36/185

(52) U.S. Cl. 424/74; 424/777

(57) ABSTRACT

A cosmetic composition comprising Moringa seed extract in combination with at least one liophilic particulate.

### RELATED APPLICATIONS

This application claims priority from provisional patent application Ser. No. 60/686,208, filed Jun. 1, 2005.

### TECHNICAL FIELD

The invention is in the field of cosmetic compositions for treating, coloring, or conditioning skin, nails, or hair.

### BACKGROUND OF THE INVENTION

In many cases, certain environmental conditions are hard on skin. For example, in large industrial cities the air tends to contain pollutants such as soot, dirt, and other particulates. These pollutants come into contact with skin and cause a number of adverse skin conditions including blemishes, skin irritation, or possibly even rosaceae. In the past, cosmetics manufacturers have tried to address the effect of pollutants on skin by formulating products that address the symptoms associated with the particular environmental assault. For example, in the case where the environmental assault results in red, irritated skin, cosmetics manufacturers have been known to include soothing herbs such as chamomile or rosemary in skin products targeted to skin having these maladies. While the herbs are very effective in treating the symptom of the environmental assault, they do not directly target the actual cause of the red, irritated skin—the soot or dirt in the air that causes the aggravation.

Certain peptides from the Morginaceae family are known to said to have anti-pollution properties. More particularly, seeds from plants of the Moringo genus are used to clarify turbid water. It is believed that these seeds are able to flocculate dirt and other particulates in the water to render the water more clear.

U.S. Pat. No. 6,500,470 is directed to preparation of extracts of Moringa seeds and use of these extracts in various types of skin creams and lotions. While the skin creams and lotions exhibit desirable properties due to the presence of the Moringa seed extract, these compositions may suffer from stability issues typically found in emulsions.

Accordingly, it is an object of the invention to provide stable emulsions containing Moringa seed extract.

It is a further object of the invention to provide cosmetic compositions containing Moringa seed extract in combination with at least one compatible lipophilic particulate.

It is a further object of the invention to provide emulsion cosmetic compositions containing Moringa seed extract in combination with at least one compatible lipophilic particulate in the oily phase of the emulsion.

It is a further object of the invention to provide a pigmented emulsion cosmetic composition containing Moringa seed extract in combination with at least one lipophilic particulate that is a pigment, powder, or mixture thereof.

It is a further object of the invention to provide an emulsion cosmetic composition containing Moringa seed extract in combination with at least one lipophilic particulate in a water and silicone oil emulsion.

### SUMMARY OF THE INVENTION

The invention is directed to a cosmetic composition containing Moringa seed extract in combination with at least one compatible lipophilic particulate.

The invention is further directed to an emulsion cosmetic composition containing Moringa seed extract and a lipophilic particulate in the oily phase of the emulsion.

The invention is further directed to a pigmented emulsion cosmetic composition cosmetic composition containing Moringa seed extract in combination with at least one lipophilic particulate that is a pigment, powder, or mixture thereof.

### DETAILED DESCRIPTION

All percentages mentioned herein are percentages by weight unless otherwise indicated.

I. Moringa Seed Extract

The cosmetic compositions of the invention contains an extract of Moringa seeds. Generally such extract in present in amounts ranging from about 0.01-80%, preferably about 0.05-75%, more preferably about 0.1-60% by weight of the total composition. Most preferred is an extract of Moringa seeds as set forth in U.S. Pat. No. 6,500,470, which is hereby incorporated by reference in its entirety. This particular extract is sold by Laboratoires Serobiologiques (a division of Cognis) and is a mixture of water, glycerin, PVP, and Moringa Plerygosperma beed Extract. This mixture is sold under the tradename Purisilk® or Pure Skin. The Moringa seed extract is generally obtained by extracting kernels of Moringa oleifera seeds by decorticating and delipidating the seeds; or crushing the seeds.

II. Lipophilic Particulate

The cosmetic composition of the invention further contains at least one lipophilic particulate in an amount ranging from about 0.01-80%, preferably about 0.5-75%, more preferably about 1-65% by weight of the total composition. Suitable lipophilic particulates may be laminar, spherical, or in other sizes or shapes. Typically such particulates have a particle size ranging from about 0.01-200 microns, preferably from about 0.05-150 microns. The lipophilic particulates may include pigments, fillers, or mixtures thereof.

The term “lipophilic” when used with particulate, means that the particulate is soluble or dispersible in the lipophilic phase of the cosmetic composition, or otherwise is compatible with the lipophilic phase. The particulate may be naturally lipophilic, or it may be naturally hydrophilic but treated with a material that confers lipophilicity to the particulate. Examples of such lipophilic particulates include iron oxides, titanium dioxides, or powders, treated with one or more hydrophobic agents such as silicone, mineral oil, esters, or similar lipophilic oils or waxes.

A. Pigments

The lipophilic particulates may include inorganic or organic pigments, or mixtures thereof.

1. Organic Pigments

Examples of organic pigment families that may be used herein include azo, (including monoazo and diazo), fluoran, xanthene, indigoid, triphenylmethane, anthroquinone, pyrene, pyrazole, quinoline, quinoline, or metallic salts thereof. Preferred are D&C colors, FD&C colors, or Lakes of D&C or FD&C colors. The term “D&C” means drug and cosmetic colors that are approved for use in drugs and cosmetics by the FDA. The term “FD&C” means food, drug, and cosmetic colors that are approved for use in foods, drugs, and cosmetics by the FDA. Certified D&C and FD&C colors are listed in 21 CFR 74.101 et seq. and include the FD&C colors Blue 1, Blue 2, Green 3, Orange B, Citrus Red 2, Red 3, Red 4, Red 40, Yellow 5, Yellow 6, Blue 1. Blue 2; Orange B. Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Suitable Lakes of D&C and FD&C colors are defined in 21 CER 82.51. Particularly preferred are Lakes formed by the reaction of the organic pigment with a metallic salt such as aluminum, calcium, zirconium, barium, and the like, Suitable reds include pigments from the monoazo, disazo, fluoran, xanthene, or indigoid families or Lakes thereof, such as Red 4, 6, 7, 17, 21, 22, 27, 28, 30, 31, 33, 34, 36, and Red 40. Also suitable are Lakes of such red pigments. Typically the metal salts are aluminum, barium, and the like.

Suitable yellows include those where the yellow pigment is a pyrazole, monoazo, fluoran, xanthene, quinoline, or salt thereof, such as Yellow 5, 6, 7, 8, 10, and 11, as well as Lakes of such yellow pigments.

Suitable violets include those from the anthroquinone family, such as Violet 2 and Lakes thereof. Examples of orange pigments are Orange 4, 5, 10, 11, or Lakes thereof.

1. Inorganic Pigments

Suitable inorganic pigments include iron oxides such as red, blue, black, green, and yellow; titanium dioxide, and the like. Preferred are iron oxides. The iron oxides may be treated with hydrophobic agents such as silicone, lecithin, mineral oil, flower or plant waxes, silicone waxes, methicone, or similar materials, will cause the pigment to be hydrophobic or lipophilic in nature, exhibiting an affinity for oily phase ingredients.

B. Particulate Fillers

The composition may contain one or more particulate fillers, which are generally non-pigmentitious powdery materials. If so, suggested ranges are about 0.001-40%, preferably about 0.05-35%, more preferably about 0.1-30% by weight of the total composition. Preferably, the particulate fillers have particle sizes ranging from about 0.02 to 100, preferably 0.5 to 100, microns. Suitable particle fillers include titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silk powder, silica, talc, mica, zinc laurate, zinc myristate, zinc rosinate, alumina, attapulgite, calcium carbonate, calcium silicate, dextran, kaolin, nylon, silica silylate, sericite, soy flour, tin oxide, titanium hydroxide, trimagnesium phosphate, walnut shell powder, or mixtures thereof. The above mentioned powders may be surface treated with lecithin, amino acids, mineral oil, silicone oil, methicone, flower, plant, or silicone waxes, or various other agents either alone or in combination, which coat the powder surface and render the particles more lipophilic in nature.

III. Other Ingredients

A. Water

The compositions of the invention may be anhydrous or in the emulsion form. If the latter, the compositions may comprise 1-95%, preferably 5-80%, more referably 10-70% by weight of the total composition of water.

B. Humectants

The composition may contain humectants, which are often soluble in the aqueous phase of the emulsion should the composition be in the emulsion form. If present, such humectants may range from about 0.001-20%, preferably from about 0.005-15%, more preferably from about 0.01-10% by weight of the total composition. Examples of such humectants include:

1. Alcohols

Suitable alcohols include mono-, di-, or polyhydric alcohols such as ethanol, propanol, butylene glycol, propylene glycol, benzyl alcohol, butyl alcohol, hexanol, and the like. If one or more of these alcohols are present in the composition, a range of 0.01-10%, preferably, 0.05-8%, more preferably 0.1-5% by weight of the total composition is suggested. Particularly preferred alcohols are propylene glycol, butylene glycol, or mixtures thereof.

C. Plant Extracts

A variety of water soluble plant extracts may be suitable for use in the composition. If present, such water soluble plant extracts are typically found in the aqueous phase, and include extracts of flowers or plants such as chamomile, aloe, apple, lady's slipper, meadowsweet, lime, lemon, lilac, oat bran, mulberry root, grapefruit, saxifraga sarmentosa, tannic acid, kojic acid, and so on. Suitable ranges of water soluble plant extracts are 0.001-8%, preferably 0.005-5%, more preferably 0.01-4% by weight of the total composition.

D. Emulsion Stabilizers

In the event the compositions are in the emulsion form, it may be desirable to include one or more emulsion stabilizers in the aqueous phase. Suitable emulsion stabilizers are salts such as potassium chloride, sodium chloride, magnesium sulfite, ammonium chloride, and the like. If present in the composition, a suitable range for the emulsion stabilizer comprises 0.001-5%, preferably 0.005-4%, more preferably 0.1-3% by weight of the total composition. The preferred emulsion stabilizer is magnesium sulfate.

E. Oils

The compositions may comprise from about 0.1-80%, preferably 1-30%, more preferably 5-25% by weight of the total composition of one or more oils. The term “oil” means an ingredient that is liquid at room temperature (25° C.). Oils include:

1. Silicone Oil

The silicone oil may be volatile, non-volatile, or a mixture of both.

(a). Volatile Silicone Oils

Suitable volatile silicones include Cyclic silicones (or cyclomethicones) are of the general formula: 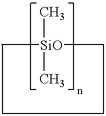
where n=3-7.

Linear volatile silicones in accordance with the invention have the general formula:
(CH₃)₃Si—O—[Si(CH₃)₂—O]ₙ—Si(CH₃)₃
where n=0-6, preferably 0-5.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise hexamethyldisiloxane, octamethyltrisiloxane, decamethyltetrasiloxane, dodecamethylhexasiloxane, octamethylcyclotetrasiloxane, decamethylcyclopentasiloxane, dodecamethylhexasiloxane, and mixtures thereof.

(b). Non-Volatile Silicone Oils

Suitable nonvolatile silicones include water insoluble silicones having a viscosity ranging from about 10 to 600,000 centistokes, preferably 20 to 100,000 centistokes at 25° C. Suitable water insoluble silicones include cetyl dimethicone, dimethicone, phenyl trimethicone, phenyldimethicone, diphenyl dimethicone, and mixtures thereof. Such silicones are available from Dow Corning as the 3225C or 5225C Formulation Aid, Dow 190 and 193 fluids, or similar products marketed by Goldschmidt under the ABIL tradename.

Also suitable as the nonvolatile silicone oil are various fluorinated silicones such as trimethylsilyl endcapped fluorosilicone oil, polytrifluoropropylmethylsiloxanes, and similar silicones such as those disclosed in U.S. Pat. No. 5,118,496 which is hereby incorporated by reference.

In one preferred embodiment of the invention, the compositions comprise a mixture of volatile and non-volatile silicones, in particular, about 0.5-40% by weight of the total composition of volatile silicone oil, and about 0.5-15% nonvolatile silicone. The presence of the volatile silicone enables the composition to dry on the skin in an appropriate period of time, and minimizes the heavy, greasy feel that is occasionally found with nonvolatile oils. The remaining nonvolatile oil phase acts to plasticize the film formed on the skin and provide a moisturizing effect to the composition.

2. Organic Oils

Also suitable for use in the composition are organic oils. The organic oils may be volatile or non-volatile.

(a). Volatile Organic Oils

Suitable volatile organic oils include various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 centipoise at 25° C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecane (having the tradename Permethyl R), are also suitable.

(b). Non-Volatile Organic Oils

Various non-volatile oils may also be suitable for use in the composition, and include hydrocarbons, esters, and the like. Suitable hydrocarons include paraffinic hydrocarbons such as isobutene, isoeicosane, isohexadecane mineral oil, squalene, squalane, and the like.

Also suitable are various esters that are liquid at room temperature. Suitable esters include guerbet esters, which are generally defined as esters that are formed by the reaction of a guerbet alcohol (which is a branched chain alcohol) having the general formula: 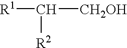
with a carboxylic acid having the general formula:
R³COOH, or
HOOC—R³—COOH, or 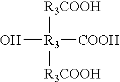
wherein R¹ and R² are each independently a C₄₋₂₀ alkyl and each R³ is a substituted or unsubstituted C₁₋₅₀ straight or branched chain alkyl or alkylene group, or phenyl, wherein the substituents are halogen, hydroxyl, carboxyl, or alkylcarbonyloxy.

Other suitable esters include those having the formula RCO—OR′ wherein R and R′ are each independently a C₁₋₂₅, preferably a C₄₋₂₀, straight or branched chain alkyl, alkenyl or alkoxycarbonylalkyl or akylcarbonyloxyalkyl. Preferred are esters that are the reaction product of of a branched chain fatty acid and a branched or straight chain fatty alcohol, preferably a branched chain fatty alcohol. Examples of such esters include isotridecyl isononanoate, isostearyl neopentanoate, tridecyl neopentanoate, cetyl octanoate, cetyl ricinoleate, decyl isostearate, isodecyl oleate, isodecyl neopentanoate, isohexyl neopentanoate, tridecyl octanoate, and so on.

Other suitable esters include naturally occuring glyceryl esters of fatty acids, or triglycerides. Both vegetable and animal sources may be used. Examples of such oils include castor oil, lanolin oil, triisocetyl citrate, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable are synthetic or semi-synthetic glyceryl esters, e.g. fatty acid mono-, di-, and triglycerides which are natural fats or oils that have been modified, for example, acetylated castor oil, glyceryl stearate, glyceryl dioleate, glyceryl distearate, glyceryl trioctanoate, glyceryl linoleate, glyceryl myristate, glyceryl isostearate, PEG castor oils, PEG glyceryl oleates, PEG glyceryl stearates, PEG glyceryl tallowates, and so on.

F. Waxes

The compositions may also contain one or more waxes which are solid or semi-solid at room temperature. Preferably, such waxes are soluble or miscible with the oil phase when the wax and the oil phase are heated together to a temperature sufficient to melt the wax. Suggested ranges of wax, if present, range from about 0.01-20%, preferably 0.1-15%, more preferably 1-10% by weight of the total composition. The waxes preferably have a melting point of about 39 to 135° C., preferably in the range of 45 to 95° C., most preferably 55 to 95° C. Suitable waxes generally include animal waxes, plant waxes, mineral waxes, silicone waxes, synthetic waxes, and petroleum waxes. More specifically, these waxes include tribehenin, bayberry, beeswax, candelilla, carnauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, cetyl alcohol, beeswax, PEC-20 sorbitan beeswax, PEL-8 beeswax, rice bran wax, shellac wax, spent grain wax, sulfurized jojoba oil, synthetic beeswax, synthetic candelilla wax, synthetic carnauba wax, synthetic japan wax, synthetic jojoba oil, synthetic wax, polyethylene, stearoxy dimethicone, dimethicone behenate, stearyl dimethicone, and the like, as well synthetic homo- and copolymer waxes such as PVP/eicosene copolymer, PVP/hexadecene copolymer, and the like. Particularly preferred is where the wax is an organic wax, tribehenin.

G. Surfactants

The compositions of the invention may comprise an effective amount of a surfactant. If the composition is in the form of an emulsion, a surfactant which is capable of causing the water phase and the oil phase to form a stable emulsion is preferably present. Suggested ranges of surfactant are in the range of from about 0.01-20%, preferably 0.1-15%, more preferably 1-10% by weight of the total composition of one or more surfactants. Suitable surfactants include organic or silicone surfactants, which may be anionic, cationic, nonionic, zwitterionic, or amphoteric. Preferably the surfactants are nonionic organic or silicone surfactants.

1. Organic Surfactants

Examples of nonionic organic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, laureth, 1-100 where the number of repeating ethylene oxide units is 1 to 100, and so on. Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol.

Also suitable as the nonionic surfactant are alkyoxylated carboxylic acids, which are formed by the reaction of a carboxylic acid with an alkylene oxide or with a polymeric ether. The resulting products have the general formula: 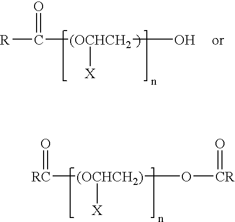
where RCO is the carboxylic ester radical, X is hydrogen or lower alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

Also suitable as the nonionic surfactant are monomeric, homopolymeric and block copolymeric ethers. Such ethers are formed by the polymerization of monomeric alkylene oxides, generally ethylene or propylene oxide. Such polymeric ethers have the following general formula:

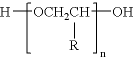

wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

2. Silicone Surfactants

Also suitable as nonionic surfactants are silicone surfactants, which are defined as silicone polymers that have at least one hydrophilic radical and at least one lipophilic radical. The silicone surfactant that may be used in the compositions of the invention are organosiloxane polymers that may be liquids or solids at room temperature. The organosiloxane surfactant is generally a water-in-oil or oil-in-water type surfactant which is, and has a Hydrophile/Lipophile Balance (HLB) of 2 to 18. Preferably the organosiloxane is a nonionic surfactant having an HLB of 2 to 12, preferably 2 to 10, most preferably 4 to 6 The HLB of a nonionic surfactant is the balance between the hydrophilic and lipophilic portions of the surfactant and is calculated according to the following formula:
HLB=7+11.7×log M_{w}/Mₒ
where M_{w} is the molecular weight of the hydrophilic group portion and Mₒ is the molecular weight of the lipophilic group portion.

The polymeric organosiloxane surfactant used in the invention may have any of the following general formulas;
MₓQ_{y}, or
MₓT_{y}, or
MDₓD′_{y}D″_{z}M
wherein each M is independently a substituted or unsubstituted trimethylsiloxy endcap unit. If substituted, one or more of the hydrogens on the endcap methyl groups are substituted, or one or more methyl groups are substituted with a substituent that is a lipophilic radical, a hydrophilic radical, or mixtures thereof. T is a trifunctional siloxy unit having the empirical formula RSiO_{1.5} or R′SiO_{1.5}. Q is a quadrifunctional siloxy unit having the empirical formula SiO₂, and D, D′, D″, x, y, and z are as set forth below, with the proviso that the compound contains at least one hydrophilic radical and at least one lipophilic radical. Preferred is a linear silicone of the formula:
MDₓD′_{y}D″_{z}M
wherein M RRRSiOl/₂

D and D′=RR′SiO_{2/2}

D″=SiO_{2/2}

x, y, and z are each independently 0-1000,

where R is methyl or hydrogen, and R′ is a hydrophilic radical or a lipophilic radical, with the proviso that the compound contains at least one hydrophilic radical and at least one lipophilic radical.

Most preferred is wherein

M=trimethylsiloxy

D=Si[(CH₃)][(CH₂)ₙCH₃]O_{2/2} where n=0-40,

D′=Si[(CH₃)][(CH₂)ₒ—O—PE)]O_{2/2} where PE is (—C₂H₄O)ₐ(—C₃H₆O)_{b}H, o=0-40,

a=1-100 and b=1-100, and

D″=Si(CH₃)₂O_{2/2}

More specifically, suitable silicone surfactants have the formula: 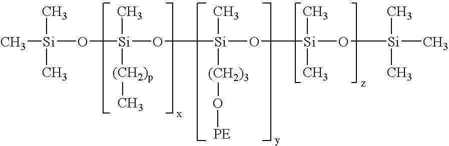
wherein p is 0-40, and
PE is (—C₂H₄O)ₐ(—C₃H₆O)_{b}—H
where x, y, z, a, and b are such that the maximum molecular weight of the polymer is approximately 50,000.

Another type of preferred organosiloxane emulsifier suitable for use in the compositions of the invention are emulsifiers sold by Union Carbide under the Silwet™ trademark, which are referred to by the CTFA term “dimethicone copolyol.

Also suitable as nonionic silicone surfactants are hydroxy-substituted silicones such as dimethiconol, which is defined as a dimethyl silicone substituted with terminal hydroxy groups.

Examples of silicone surfactants are those sold by Dow Coring under the tradename Dow Corning 3225C or 5225C Formulation Aid, Dow Corning 190 Surfactant, Dow Corning 193 Surfactant, Dow Corning Q2-5200, and the like are also suitable. In addition, surfactants sold under the tradename Silwet by Union Carbide, and surfactants sold by Troy Corporation under the Troysol tradename, those sold by Taiwan Surfactant Co. under the tradename Ablusoft, those sold by Hoechst under the tradename Arkophob, are also suitable for use in the invention.

In one preferred embodiment of the invention, the surfactant is selected from an nonionic organic surfactant, in particular, an alkoxylated alcohol; a silicone surfactant; and mixtures thereof.

H. Preservatives

The composition may contain 0.0001-8%, preferably 0.001-6%, more preferably 0.005-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, propyl paraben, methyl paraben, benzoic acid, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

I. Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin F and derivatives thereof such as Vitamin E acetate, nicotinate, or other esters thereof. In addition. Vitamins D, C, and K, as well as derivatives thereof are suitable. Particularly preferred are derivatives of vitamins C, F, and A such as magnesium ascorbyl phosphate, retinyl palmitate, tocopheryl acetate, and mixtures thereof.

Suitable antioxidants are ingredients that assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

J. Sunscreens

Suitable sunscreens include chemical or physical sunscreen. Chemical sunscreens include those that absorb in the UVA and UVB range. If present, the sunscreens may range from about 0.001-65%, preferably from about 0.005-50% more preferably from about 0.01-35% by weight of the total composition.

1. UVA Chemical Sunscreens

The term “UVA sunscreen” means a chemical compound that blocks UV radiation in the wavelength range of about 3220 to 400 nm. One type of preferred UVA sunscreen are dibenzoylmethane compounds having the general formula: 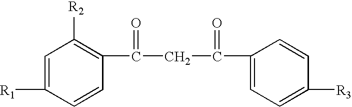
wherein R₁ is H, OR and NRR wherein each R is independently H, C₁₋₂₀ straight or branched chain alkyl; R₂ is H or OH; and R₃ is H, C₁₋₂₀ straight or branched chain alky.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Also suitable are triazine sunscreen agents sold under the trade name Tinosorb®. Examples are Tinosorb® S, having the name ethylhexyloxyphenol methoxyphenyl triazine, and Tinosorb® M, which has the chemical name methylene bis-benzotriaolyl tetramethylbutylphenol.

Further examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylniethane, 4,4′diisopropylbenzoylmethane, 4-tert-butyl-4′-methoxydibenzoylmethane, 4,4′-diisopropylbenzoylmethane, 2-methyl-5-isorpoyl-4′-methoxydibenzoymethane, 2-metyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

The claimed compositions may contain from about 0.001-20%, preferably 0.005-5%, more preferably about 0.005-3% by weight of the composition of UVA sunscreen. In one preferred embodiment of the invention the UVA sunscreen is Avobenzone, and it is present at not greater than about 3% by weight of the total composition.

2. UVB Chemical Sunscreens

The term “UVB sunscreen” means a compound that blocks UV radiation in the wavelength range of from about 290 to 320 nm. A variety of UVB chemical sunscreens exist including α-cyano-β,β-diphenyl acrylic acid esters as set forth in U.S. Pat. No. 3,215,724, which is hereby incorporated by reference in its entirety. One particular example of a α-cyano-β,β-diphenyl acrylic acid ester is Octocrylene, which is 2-ethylhexyl 2-cyano-3,3-diphenylacrylate. In certain cases the composition may contain no more than about 10% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula: 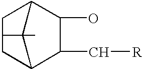
wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula: 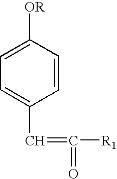
wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at no more than about 3% by weight of the total composition.

Also suitable as the UVB screening agents are various benzophenone derivatives having the general formula: 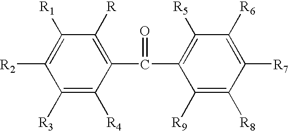
R through R₉ are each independently H, OH, NaO₃S, SO₃H, SO₃Na, Cl, R″, OR″ where R″ is C₁₋₂₀ straight or branched chain alkyl. Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone) and Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula: 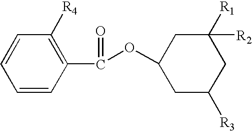
wherein R₁, R₂, R₃, and R₄ are each independently X, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homnosalate is available commercially from Merck under the tradename Eusolex HMS and menthyl anthranilate is commercially available from Haarmann & Reimer under the tradename Heliopan. If present the Homosalate should be found at no more than about 15% by weight of the total composition.

Various amino benzoic acid derivatives are suitable UVB absorbers including those having the general formula: 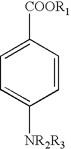
Wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alky, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula: 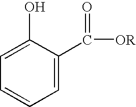
wherein R is a straight or branched chain alkyl, including derivatives of the above compound formed from mono-, di-, or triethanolamines. Particular preferred are octyl salicylate, TEA-salicylcate, DEA-salicylate and mixtures thereof.

Generally, the amount of the UVB chemical sunscreen present may range from about 0.001-45%, preferably 0.005-40%, more preferably about 0.01-35% by weight of the total composition.

The invention will be further described in connection with the following examples that are set forth for the purposes of illustration only.

### EXAMPLE 1

Facial makeup compositions were made as follows:

| | % by weight | |
|---|---|---|
| | | Face |
| Ingredient | Foundation | Shimmer |
| Cetyl dimethicone copolyol | 2.00 | 2.50 |
| Dimethicone copolyol | 16.25 | — |
| Polyglyceryl-4 isostearate | — | 2.25 |
| Octinoxate | — | 7.50 |
| Isotridecyl isononanoate | — | 4.00 |
| Sorbitan sesquioleate | 0.05 | — |
| Tocopheryl acetate | 0.05 | 0.05 |
| Retinyl palmitate | 0.05 | 0.01 |
| Polyglyceryl-3 diisostearate | — | 1.00 |
| Propyl paraben | 0.10 | 0.10 |
| Boron nitride | — | 1.78 |
| Titanium dioxide, lauric acid, aluminum | — | 3.00 |
| hydroxide | | |
| Titanium dioxide, alumina, methicone | 5.00 | — |
| Titanium dioxide, methicone | 2.00 | — |
| Zinc oxide, methicone | 2.20 | — |
| Nylon 12 | 2.00 | — |
| Alumina | 0.30 | — |
| Talc, methicone | 2.30 | — |
| Boron nitride | 0.70 | — |
| HDI/trimethylol hexyllactone crosspolymer, | 2.15 | — |
| silica | | |
| Acrylonitrile/methacrylonitrile/methyl | 0.05 | — |
| methacrylate copolymer, iron oxides, talc, | | |
| water | | |
| Iron oxides/boron nitride | — | 0.44 |
| Iron oxides/methicone | — | 0.40 |
| Mica, methicone | — | 1.67 |
| Dimethicone | 2.00 | 2.00 |
| Iron oxides, galactoarabinan | 1.30 | — |
| Phenoxyethanol | 0.70 | 0.70 |
| Cyclomethicone | 11.25 | 14.3 |
| Dimethicone, octamethyltrisiloxane | 3.00 | — |
| Cyclomethicone, trimethylsiloxysilicate | — | — |
| (50:50) | | |
| Cyclomethicone, dimethicone, phenyl | — | 2.00 |
| trimethicone, polyethylene | | |
| Cyclomethicone, dimethicone, ammonium | 1.50 | — |
| polyacryloyldimethyl taurate, polysorbate 20, | | |
| polysorbate 80, tocopheryl acetate | | |
| Cyclomethicone, Gingko biloba extract, Panax | 0.25 | — |
| ginseng root extract, Camellia Sinensis leaf | | |
| extract, Centaurea Cyanus flower extract, Vitis | | |
| Vinefera seed extract | | |
| Trihydroxystearin | — | 0.55 |
| Tribehenin | 0.05 | — |
| Mica, titanium dioxide, iron oxides, methicone | — | 2.60 |
| Mica, titanium dioxide, methicone | — | 1.40 |
| Water | QS | QS |
| Magnesium sulfate | — | 1.00 |
| Sodium chloride | 1.00 | — |
| Tetrasodium EDTA | 0.01 | — |
| Sodium ascorbyl phosphate | — | 0.01 |
| Diazolidinyl urea | — | 0.20 |
| Aloe barbadensis leaf juice | 0.05 | 0.10 |
| Sodium hyaluronate, hydrolyzed | 0.20 | — |
| glycosaminoglycans | | |
| Water, glycerin, PVP, Moringa Pterygosperma | 0.25 | 0.25 |
| Seed Extract | | |
| Sodium hyaluronate | — | 0.01 |
| Glycerin | — | 3.00 |
| Butylene glycol | 3.00 | 2.00 |
| Methyl paraben | 0.20 | 0.25 |
| Ethyl paraben | 0.15 | — |
| Cyclomethicone, dimethiconol | — | 2.00 |
| Mica, methicone | — | 0.01 |
| Cyclomethicone, disteardimonium hectorite, | 6.00 | — |
| alcohol | | |

The compositions were prepared by grinding the pigments in a portion of the oils. Separately, the oily phase ingredients were combined and mixed with the pigment grind. The oils were emulsified in the water and water soluble ingredients.

### EXAMPLE 2

Skin care compositions were prepared as follows:

| | % by weight | | | | |
|---|---|---|---|---|---|
| | | | | | Body Firming |
| Ingredient | Eye Cream | Day Lotion | Skin Serum | Day Cream | Cream |
| Water | QS | QS | QS | QS | QS |
| Disodium EDTA | 0.10 | 0.10 | — | 0.18 | — |
| Tetrasodium EDTA | — | — | — | — | 0.05 |
| Triethanolamine | — | 1.175 | — | 1.00 | 1.43 |
| Magnesium ascorbyl phosphate | 0.05 | 0.10 | 0.001 | 0.10 | 0.001 |
| Imidazolidinyl urea | — | — | 0.30 | — | — |
| Potassium sorbate | 0.20 | — | — | 0.20 | — |
| Allantoin | — | — | — | — | 0.50 |
| Hydroxyethyl cellulose | 0.05 | — | — | — | — |
| Xanthan gum | — | — | 0.25 | — | 0.25 |
| Butylene glycol | 5.00 | 3.00 | 5.30 | 5.00 | 2.00 |
| Methyl paraben | 0.25 | 0.25 | 0.20 | 0.36 | 0.30 |
| Hydrolyzed Oat Flour | 0.20 | — | — | — | — |
| Mica, titanium dioxide | — | — | 0.35 | 1.00 | — |
| Silica | — | — | 0.80 | 0.75 | — |
| Talc | — | — | — | 0.75 | — |
| Caprylic/capric myristic/stearic | 1.50 | — | — | — | — |
| triglyceride | | | | | |
| Tridecyl stearate, neopentyl glycol | — | — | — | — | 3.50 |
| dicaprylate/dicaprate, tridecyl | | | | | |
| trimellitate | | | | | |
| Petrolatum | — | — | — | — | 4.00 |
| Glyceryl stearate | — | — | — | — | 1.20 |
| PEG-40 stearate | — | — | — | — | 1.40 |
| Isohexadecane | — | — | — | — | 2.00 |
| Isononyl isononanoate | — | — | — | — | 5.00 |
| Butylene glycol dicaprylate/dicaprate | — | — | — | 6.00 | — |
| Cetyl alcohol | — | — | — | 1.50 | 0.60 |
| Stearyl alcohol | — | — | — | 0.75 | — |
| Octinoxate | — | 7.50 | — | 1.50 | — |
| Ethylhexyl methoxy-cinnamate | — | — | — | 7.50 | — |
| Oxybenzone | — | 4.00 | — | 4.00 | — |
| Avobenzone | — | 2.00 | — | 2.00 | — |
| Lauryl lactate | — | 1.50 | — | 1.50 | — |
| PEG-100 stearate | — | — | — | 0.75 | — |
| Stearic acid | — | 0.50 | — | — | — |
| Octisalate | — | 5.00 | — | — | — |
| Ethyl paraben | 0.15 | 0.15 | — | 0.15 | 0.10 |
| Dimethicone | 1.50 | 1.00 | 1.00 | 3.00 | — |
| Propyl paraben | 0.10 | 0.10 | — | 0.10 | 0.10 |
| Steareth-2 | 1.50 | 1.00 | — | — | — |
| Steareth-21 | 1.85 | 0.50 | — | — | — |
| Polysorbate 60 | — | — | — | 2.60 | — |
| PPG-2 myristyl ether propionate | — | — | — | 4.50 | — |
| Sorbitan stearate | — | — | — | 0.90 | — |
| C12-15 alkyl benzoate | — | — | — | 2.00 | — |
| Behenyl alcohol | 2.00 | — | — | — | — |
| Tocopheryl acetate | 0.20 | 0.10 | 0.10 | 0.20 | 0.10 |
| Jojoba seed oil | — | 0.10 | — | — | 0.05 |
| Octyldodecyl neopentanoate | 3.00 | 1.00 | — | — | — |
| Propylene glycol stearate | 2.00 | — | — | — | — |
| Retinyl palmitate | 0.05 | 0.001 | 0.005 | 0.05 | 0.001 |
| Microcrystalline wax | 1.50 | — | — | — | — |
| Phenoxyethanol | 1.00 | 1.00 | — | 0.9 | 1.00 |
| Liquid paraffin, coconut oil, aloe | 0.10 | — | — | — | 0.05 |
| barbadensis leaf extract | | | | | |
| Liquid paraffin | — | — | 1.50 | — | — |
| Jojoba seed oil | 0.10 | — | — | 0.10 | — |
| Polyethylene | 0.10 | — | — | — | — |
| Betula Alba extract | 0.05 | 0.05 | — | 0.05 | 0.05 |
| Hydrogenated polyisobutene | 1.00 | — | 1.50 | — | — |
| Silica | 1.00 | — | — | — | — |
| Polyacrylamide, C13-14 isoparaffin, | 2.00 | — | 1.00 | — | — |
| laureth-7 | | | | | |
| Glyceryl polyacrylate, dimethiconol, | — | — | — | — | 2.00 |
| cyclomethicone | | | | | |
| Acrylates/C10-30 alkyl acrylate | — | 0.30 | 0.25 | — | — |
| crosspolymer | | | | | |
| Carbomer solution (2.5%) | — | 10.00 | — | 20.00 | 30.00 |
| Glycerin | 3.00 | 3.50 | — | 5.00 | 2.00 |
| Titanium dioxide | 0.30 | — | — | — | — |
| Hydrolyzed | — | — | — | — | 1.00 |
| glycosamino-glycans | | | | | |
| Soybean protein | — | — | — | — | 1.50 |
| Sodium hyaluronate, | 0.50 | — | — | — | — |
| hydrolyzed glycosamino-glycans | | | | | |
| Panthenol | 0.10 | 0.10 | — | 0.05 | — |
| Aloe Barbadensis leaf juice | — | 0.10 | — | — | — |
| Water, glycerin, Macrocystis | 5.00 | 1.00 | 1.00 | — | — |
| Pyrifera extract, hydrolyzed | | | | | |
| wheat protein, PVP | | | | | |
| Aspartic acid, glutamic acid, | — | 0.10 | — | — | — |
| alanine, dextrin, urea, sucrose, | | | | | |
| fructose, glucome, hexylene glycol | | | | | |
| Water, Salix Nigra extract | 0.50 | 1.00 | — | 1.00 | 0.50 |
| Water, glycerin, PVP, Moringa | 1.00 | 1.00 | 1.00 | 1.00 | 1.00 |
| Pterygosperma seed extract | | | | | |
| Water, saccharomyces zinc, copper, | 0.01 | 0.05 | — | 0.01 | 0.01 |
| manganese, iron, silicon, | | | | | |
| potassium, ferment | | | | | |
| Hydrolyzed wheat protein/ | 0.50 | — | — | — | — |
| PVP crosspolymer | | | | | |
| Macrocystis Pyrifera extract | 0.20 | — | — | — | — |
| Salvia Sclarea extract, Ferula | 0.05 | 0.05 | — | 0.10 | 0.30 |
| Galbaniflua gum extract, Anthemis | | | | | |
| Nobilis flower extract, Camellia | | | | | |
| Sinesis leaf extract, | | | | | |
| phenoxyethanol, Rosmarinus | | | | | |
| officinalis leaf extract | | | | | |
| Methoxypropyl-gluconamide/water | 0.50 | 0.50 | 25.00 | 0.50 | 0.50 |
| Salicylic acid/hydrolyzed vegetable | — | — | 3.50 | — | 0.10 |
| protein | | | | | |
| Glycerin, butylene glycol, water, | — | — | — | — | 1.00 |
| carbomer, polysorbate 20, palmitoyl | | | | | |
| oligo-peptide, palmitoyl tetra-peptide | | | | | |

The compositions were prepared by combining the oil phase ingredients and mixing well. Separately, water and the water phase ingredients were combined and mixed well. The oil and water phases were combined and emulsified. The compositions were poured into jars or bottles.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A cosmetic composition comprising Moringa seed extract in combination with at least one lipophilic particulate.

2. The composition of claim 1 wherein the Moringa seed extract is from Morgina Pterygosperma.

3. The composition of claim 1 wherein the cosmetic composition is anhydrous.

4. The composition of claim 1 wherein the cosmetic composition is an emulsion.

5. The composition of claim 4 wherein the emulsion is a skin lotion, cream, or serum.

6. The composition of claim 4 wherein the emulsion is a foundation makeup, concealer, bronzer, or blush.

7. The composition of claim 1 which is a water in oil emulsion foundation makeup or concealer composition comprising water, at least one silicone oil, and at least one lipophilic particulate which is a pigment.

8. The composition of claim 7 comprising 1-95% water, 0.1-80% oil, and 0.01-80% of at least one lipophilic particulate.

9. The composition of claim 8 wherein the oil comprises a volatile silicone oil, a non-volatile silicone oil, or mixtures thereof.

10. The composition of claim 8 wherein the oil comprises a mixture of volatile and non-volatile silicone oils.

11. The composition 9 wherein the volatile silicone oils as hexamethyldisiloxane, octamethyltrisiloxane, decamethyltetrasiloxane, dodecamethylpentasiloxane, octamethylcyclotetrasiloxane, decamethylcyclopentasiloane, dodecamethylcyclohexasiloxane, or mixtures thereof.

12. The composition of claim 11 wherein the volatile silicone is decamethylcyclopentasiloxane, octamethyltrisiloxane, decamethyltetrasiloxane, or mixtures thereof.

13. The composition of claim 1 wherein the lipophilic particulate comprises pigments, powders, or mixtures thereof.

14. The composition of claim 13 wherein the lipophilic particulate comprises methicone treated iron oxides.

15. The composition of claim 13 wherein the lipophilic particulate comprises methicone treated powders.

16. The composition of claim 13 wherein the lipophilic particulate comprises a mixture of pigments and powder wherein at least some of the pigments are iron oxides.

17. The composition of claim 1 which is an anhydrous pigmented cosmetic composition which is blush, eyeshadow, lipstick, or concealer.

18. The composition of claim 1 additionally comprising one or more chemical sunscreens.

19. The composition of claim 1 additionally comprising one or more plant extracts.

20. The composition of claim 1 additionally comprising one or more film forming polymers.
